(19) United States
(12) Patent Application Publication
(10) Pub. No.: US 2024/0210159 A1
(43) Pub. Date: Jun. 27, 2024

(54) OPTICAL COHERENCE TOMOGRAPHY COMMON-PATH PROBE

(71) Applicant: INDUSTRIAL TECHNOLOGY RESEARCH INSTITUTE, Hsinchu (TW)

(72) Inventors: Chy Lin WANG, Hsinchu County (TW); Chi Shen CHANG, Zhubei City (TW); Yuan Chin LEE, Hsinchu City (TW)

(73) Assignee: INDUSTRIAL TECHNOLOGY RESEARCH INSTITUTE, Hsinchu (TW)

(21) Appl. No.: 18/086,427

(22) Filed: Dec. 21, 2022

(51) Int. Cl. G01B 9/02091

(52) U.S. Cl. CPC G01B 9/02091

(57) ABSTRACT

An OCT common-path probe for identifying a sample includes an optical fiber, a first GRIN lens and a second GRIN lens. The optical fiber outputs a light beam from its end facet. The first GRIN lens is cemented to the second GRIN lens, and located between the optical fiber and the second GRIN lens. A joint surface between the first GRIN lens and the second GRIN lens reflects a part of the light beam to form a reference beam, and allows another part of the light beam to pass through to form a sample beam. The reference beam is focused on the end facet by the first GRIN lens. The sample beam is focused on the sample by the second GRIN lens, reflected by the sample to travel through the second GRIN lens, the joint surface and the first GRIN lens sequentially, and thereby focused on the end facet.

### TECHNICAL FIELD

This disclosure relates to an optical coherence tomography common-path probe.

### BACKGROUND

Conventionally, there are two light beams in an optical coherence tomography (OCT) system, one of them is a sample beam, and the other is a reference beam. When path lengths of the two light beams are equal to each other, there would be interference signals generated for identifying tissues or samples.

A fiber optic OCT probe can be used for imaging of trachea or brain tissue. However, during operation, an optical path difference between the two light beams may be varied due to movement or bending of the optical fiber, thereby resulting in bad image quality or failures in imaging. Moreover, due to manufacturing deviations, there are some degrees of differences between the lengths of the optical fibers of the manufactured probes. Therefore, when the probe is replaced with a new one, it is necessary to readjust the reference arm where the reference beam travels for the new probe, thus wasting more time and human resources.

To address the above problem, a common-path probe is developed for preventing optical path difference between the two light beams from being varied due to movement or bending of the optical fiber. However, in general, a conventional common-path probe is provided with only one gradient index lens (GRIN lens), and a reflection surface is a lens surface of the GRIN lens located farther away from the optical fiber. In such configuration, it is difficult to ensure that the sample beam and the reference beam can be both focused on an end facet of the optical fiber. For example, it is required to obtain a balance among parameters, such as the refractive index of the GRIN lens, the length of the GRIN lens and the distance between the GRIN lens and the optical fiber, so as to ensure the focusing of the sample beam on the end facet of the optical fiber. However, it is difficult to ensure that the reference beam can be focused on the end facet of the optical fiber at the same time, and usually, the reflected reference beam would converge and then diverge before it reaches the end facet of the optical fiber. Therefore, the focusing spot of the reference beam is not located on the end facet of the optical fiber, so the reference beam collection efficiency is low, and the coupling efficiency of the optical fiber is merely about 15%.

### SUMMARY

One embodiment of the disclosure provides an optical coherence tomography common-path probe for identifying a sample, and the optical coherence tomography common-path probe includes an optical fiber, a first GRIN lens and a second GRIN lens. The optical fiber has an end facet, and the optical fiber is configured to output a light beam through the end facet. The first GRIN lens is located on one side of the end facet, and the first GRIN lens is configured to collimate the light beam. The second GRIN lens is cemented to the first GRIN lens, the first GRIN lens is located between the end facet of the optical fiber and the second GRIN lens, and the second GRIN lens is configured to focus a sample beam. In addition, a joint surface is located between the first GRIN lens and the second GRIN lens. After the light beam output from the optical fiber is collimated by the first GRIN lens, a part of the light beam is reflected by the joint surface to form a reference beam, and another part of the light beam passes through the joint surface to form the sample beam entering the second GRIN lens. The reference beam is focused on the end facet of the optical fiber by the first GRIN lens. The sample beam is focused on the sample by the second GRIN lens and is reflected by the sample so as to travel through the second GRIN lens, the joint surface and the first GRIN lens sequentially, and thereby focused on the end facet of the optical fiber.

### BRIEF DESCRIPTION OF THE DRAWINGS

FIG. 1 is a schematic view of an optical coherence tomography common-path probe and a sample in accordance with one embodiment of the disclosure.

FIG. 2 is a front view of a joint surface in FIG. 1.

FIG. 3 is a schematic view of a light path of a reference beam in the optical coherence tomography common-path probe in FIG. 1.

FIG. 4 is a schematic view of a light path of a sample beam in the optical coherence tomography common-path probe in FIG. 1.

FIG. 5 is a front view of the joint surface in FIG. 1 according to another configuration of the disclosure.

FIG. 6 is a front view of the joint surface in FIG. 1 according to still another configuration of the disclosure.

### DETAILED DESCRIPTION

Aspects and advantages of the application will become apparent from the following detailed descriptions with the accompanying drawings. For purposes of explanation, one or more specific embodiments are given to provide a thorough understanding of the application, and which are described in sufficient detail to enable one skilled in the art to practice the described embodiments. It should be understood that the following descriptions are not intended to limit the embodiments to one specific embodiment. On the contrary, it is intended to cover alternatives, modifications, and equivalents as can be included within the spirit and scope of the described embodiments as defined by the appended claims.

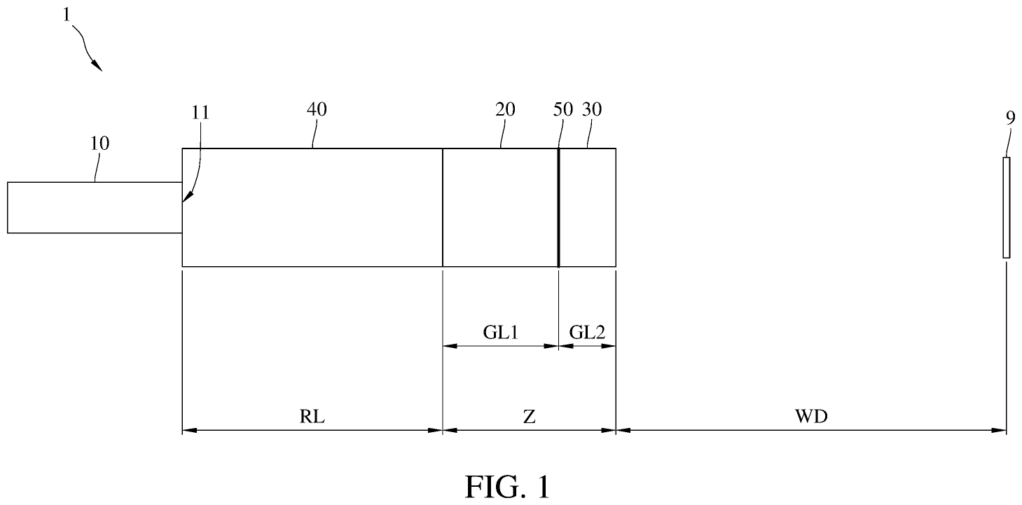

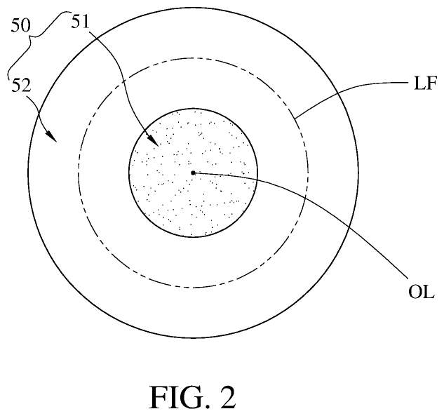

Please refer to FIG. 1 and FIG. 2. FIG. 1 is a schematic view of an optical coherence tomography common-path probe and a sample in accordance with one embodiment of the disclosure, and FIG. 2 is a front view of a joint surface in FIG. 1.

In this embodiment, an optical coherence tomography (OCT) common-path probe 1 is for identifying a sample 9, and the OCT common-path probe 1 includes an optical fiber 10, a first GRIN lens 20, a second GRIN lens 30 and a rod lens 40.

The optical fiber 10 has an end facet 11, and the optical fiber 10 is configured to output a light beam LF through the end facet 11. The light beam LF is split into a reference beam LR and a sample beam LS. In one exemplary example, a wavelength of the light beam LF may be larger than or equal to 880 nm and smaller than or equal to 1550 nm. However, in other exemplary examples, the light beam LF may have different wavelength according to actual requirements, and the present disclosure is not limited to the above disclosed examples.

The first GRIN lens 20 is located on one side of the end facet 11, and the first GRIN lens 20 is configured to collimate the light beam LF. The second GRIN lens 30 is cemented to the first GRIN lens 20, the first GRIN lens 20 is located between the end facet 11 of the optical fiber 10 and the second GRIN lens 30, and the second GRIN lens 30 is configured to focus the sample beam LS of the light beam LF. In addition, optical axes of the first GRIN lens 20 and the second GRIN lens 30 overlap, which can be considered as an optical axis OL.

The rod lens 40 is located between the first GRIN lens 20 and the end facet 11 of the optical fiber 10 and connected to the first GRIN lens 20 and the end facet 11 of the optical fiber 10.

There is a joint surface 50 located between the first GRIN lens 20 and the second GRIN lens 30. After the light beam LF output from the optical fiber 10 is collimated by the first GRIN lens 20, a part of the light beam LF is reflected by the joint surface 50 to form the reference beam LR, and another part of the light beam LF passes through the joint surface 50 to form the sample beam LS entering the second GRIN lens 30.

In one exemplary example, the joint surface 50 has a partial reflection region 51 and a light transmission region 52. The partial reflection region 51 of the joint surface 50 faces toward the end facet 11 of the optical fiber 10, and the optical axis OL of the first GRIN lens 20 and the second GRIN lens 30 is perpendicular to the partial reflection region 51. The partial reflection region 51 of the joint surface 50 is configured for a part of the light beam LF reaching the partial reflection region 51 to pass through the joint surface 50, and for another part of the light beam LF reaching the partial reflection region 51 to be reflected. The light transmission region 52 of the joint surface 50 is configured for the light beam LF reaching the light transmission region 52 to pass through and enter the second GRIN lens 30. Under the effect of the joint surface 50, the light beam LF reflected by the joint surface 50 forms the reference beam LR, and the light beam LF passing through the joint surface 50 forms the sample beam LS. In addition, the sample beam LS includes a part of the light beam LF reaching the partial reflection region 51 and the light beam LF reaching the light transmission region 52.

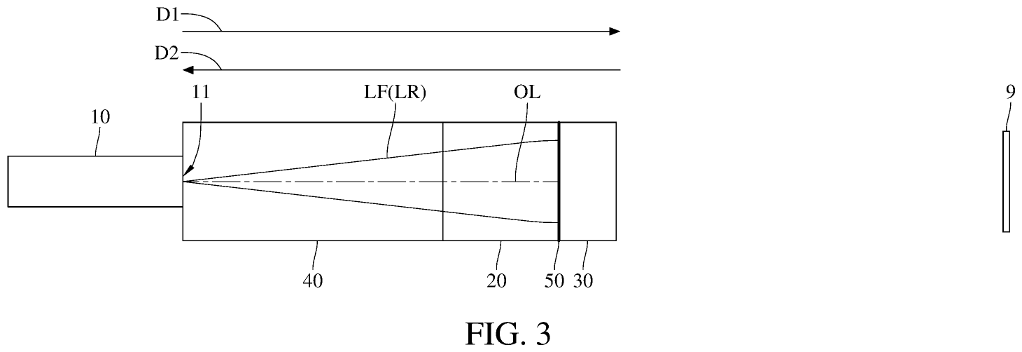

Please refer to FIG. 3, which is a schematic view of a light path of a reference beam in the optical coherence tomography common-path probe in FIG. 1. The light beam LF output from the end facet 11 of the optical fiber 10 sequentially travels through the rod lens 40 and the first GRIN lens 20 towards the joint surface 50, where a part of the light beam LF reaches the partial reflection region 51 of the joint surface 50, and is reflected by the partial reflection region 51 of the joint surface 50 to form the reference beam LR. Then, the reference beam LR travels through the first GRIN lens 20 and the rod lens 40 again and is focused on the end facet 11 of the optical fiber 10. That is, a light path route of the reference beam LR starts from the end facet 11 of the optical fiber 10 in a departure direction D1, traveling through the rod lens 40 and the first GRIN lens 20 in sequence, reaching the partial reflection region 51 of the joint surface 50, and then traveling back from the partial reflection region 51 of the joint surface 50 in a return direction D2 through the first GRIN lens 20 and the rod lens 40 in sequence to the end facet 11 of the optical fiber 10. Hence, one can see that the reference beam LR undergoes reflection only once in the light path route.

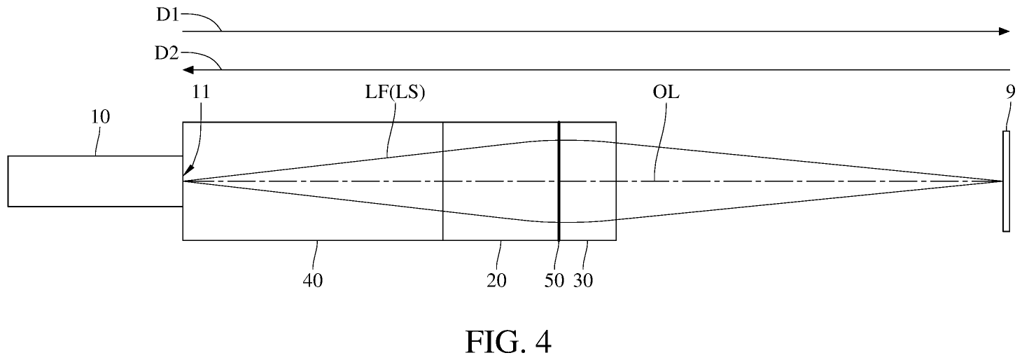

Please refer to FIG. 4, which is a schematic view of a light path of a sample beam in the optical coherence tomography common-path probe in FIG. 1. The light beam LF output from the end facet 11 of the optical fiber 10 sequentially travels through the rod lens 40 and the first GRIN lens 20 towards the joint surface 50, where a part of the light beam LF passes through the partial reflection region 51 and the light transmission region 52 of the joint surface 50 to form the sample beam LS. The sample beam LS is focused on the sample 9 by the second GRIN lens 30 and is reflected by the sample 9, and then travels through the second GRIN lens 30, the joint surface 50, the first GRIN lens 20 and the rod lens 40 sequentially and is focused on the end facet 11 of the optical fiber 10. That is, a light path route of the sample beam LS starts from the end facet 11 of the optical fiber 10 in the departure direction D1, traveling through the rod lens 40, the first GRIN lens 20, the joint surface 50 and the second GRIN lens 30 in sequence to reach the sample 9, and then traveling back from the sample 9 in the return direction D2 through the second GRIN lens 30, the joint surface 50, the first GRIN lens 20 and the rod lens 40 in sequence to the end facet 11 of the optical fiber 10.

In this embodiment, a maximum cross-section of the light beam LF in a light traveling path is located at the joint surface 50, and the light beam LF is a parallel light beam parallel to the optical axis OL at the joint surface 50; that is, the light beam LF is collimated by the first GRIN lens 20 to be a collimated light beam. Furthermore, an area of the partial reflection region 51 is smaller than a cross-sectional area of the light beam LF on the joint surface 50. In addition, according to required energy of the sample beam LS, the area of the partial reflection region 51 may be designed to be larger than or equal to 20% of the cross-sectional area of the light beam LF on the joint surface 50, and a reflectivity of the partial reflection region 51 may be designed to be larger than or equal to 4% and smaller than 100%.

In this embodiment, the area of the partial reflection region 51 is larger than 20% of the cross-sectional area of the light beam LF on the joint surface 50, but the disclosure is not limited thereto. In other embodiments, an area of a partial reflection region may be equal to 20% of a cross-sectional area of a light beam on a joint surface.

In this embodiment, the partial reflection region 51 of the joint surface 50 includes a coating layer having a reflectivity larger than or equal to 4% and smaller than 100%, but the disclosure is not limited thereto. In other embodiments, a partial reflection region of a joint surface and a GRIN lens may have certain refractive indices different from each other, so that when a light beam reaches an interface between them, both reflection and refraction of the light beam may occur, where the reflection occurs at the interface is referred as Fresnel reflection.

In this embodiment, the optical axis OL of the first GRIN lens 20 and the second GRIN lens 30 intersects the partial reflection region 51; that is, the partial reflection region 51 is located in an inner circular area of the joint surface 50 including the center (i.e., the optical axis OL) of the joint surface 50, and the light transmission region 52 is located in an outer circular area of the joint surface 50. However, in other embodiments, positions of a partial reflection region and a light transmission region may be switched; that is, the partial reflection region may be located in an outer circular area of the joint surface, and the light transmission region may be located in an inner circular area of the joint surface including the center (i.e., the optical axis) of the joint surface. In this embodiment, the partial reflection region 51 and the light transmission region 52 being circular is only exemplary, and the present disclosure is not limited to the above disclosed examples.

In this embodiment, the light beam LF passes through both of the partial reflection region 51 and the light transmission region 52 at the joint surface 50, but the disclosure is not limited thereto. In other embodiments, when a cross-sectional area of a light beam at a joint surface is smaller than a partial reflection region of the joint surface, the light beam may only reach the partial reflection region but not a light transmission region, and split into a reference beam and a sample beam under the reflection and transmission effect of the partial reflection region.

The rod lens 40 in this embodiment is optional, and the present disclosure is not limited thereto. In one exemplary example, there may not be a rod lens located between a first GRIN lens and an optical fiber. In another exemplary example, there may be other components, such as a coreless optical fiber, but not a rod lens disposed between a first GRIN lens and an optical fiber.

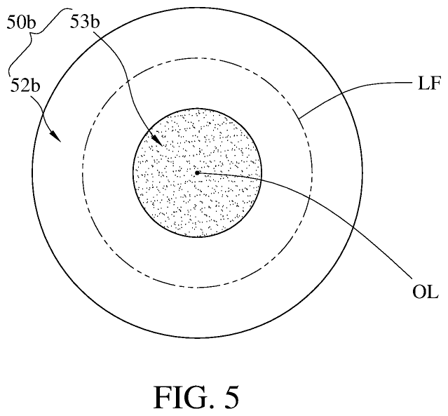

In the above embodiment, the joint surface 50 has the partial reflection region 51 and the light transmission region 52, and both of the partial reflection region 51 and the light transmission region 52 allow a part of the light beam LF to pass therethrough to form the sample beam LS, but the disclosure is not limited thereto. For example, please refer to FIG. 5, which is a front view of the joint surface in FIG. 1 according to another configuration of the disclosure. In this configuration, a joint surface 50b has a total reflection region 53b and a light transmission region 52b. The total reflection region 53b of the joint surface 50b faces toward the end facet 11 of the optical fiber 10, an area of the total reflection region 53b is smaller than a cross-sectional area of the light beam LF on the joint surface 50b, and the optical axis OL of the first GRIN lens 20 and the second GRIN lens 30 is perpendicular to the total reflection region 53b. In the arrangement of this configuration, a part of the light beam LF output from the end facet 11 travels toward the total reflection region 53b of the joint surface 50b, and is reflected by the total reflection region 53b to form the reference beam LR. The reference beam LR is focused on the end facet 11 of the optical fiber 10 by the first GRIN lens 20. Another part of the light beam LF output from the end facet 11 passes through the light transmission region 52b of the joint surface 50b and enters the second GRIN lens 30 to form the sample beam LS. The sample beam LS is focused on the sample 9 by the second GRIN lens 30 and is reflected by the sample 9, traveling through the second GRIN lens 30, the light transmission region 52b of the joint surface 50b and the first GRIN lens 20 sequentially, and then focused on the end facet 11 of the optical fiber 10. In addition, a reflectivity of the total reflection region 53b is equal to 100%, and the total reflection region 53b may be coated with, for example, a coating layer having a reflectivity equal to 100%. In addition, according to required energy of the sample beam LS, the area of the total reflection region 53b may be designed to be larger than or equal to 20% of the cross-sectional area of the light beam LF on the joint surface 50b, and the present disclosure is not limited to the area ratio as shown in FIG. 5.

In this embodiment, the optical axis OL of the first GRIN lens 20 and the second GRIN lens 30 intersects the total reflection region 53b; that is, the total reflection region 53b is located in an inner circular area of the joint surface 50b where the center (i.e., the optical axis OL) thereof is located, and the light transmission region 52b is located in an outer circular area of the joint surface 50b. However, in other embodiments, positions of a total reflection region and a light transmission region may be switched; that is, the total reflection region may be located in an outer circular area of the joint surface, and the light transmission region may be located in an inner circular area of the joint surface 50b including the center (i.e., the optical axis OL) of the joint surface 50b. In this embodiment, the total reflection region 53b and the light transmission region 52b being circular is only exemplary, and the present disclosure is not limited to the above disclosed examples.

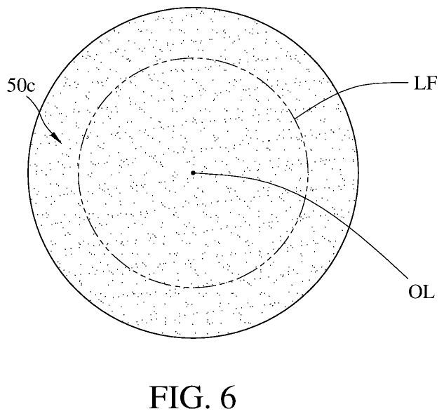

As another example, please refer to FIG. 6, which is a front view of the joint surface in FIG. 1 according to still another configuration of the disclosure. In this configuration, a joint surface 50c is entirely a partial reflection surface and faces towards the end facet 11 of the optical fiber 10. The partial reflection surface is configured for a part of the light beam LF to be reflected to form the reference beam LR, and for another part of the light beam LF to pass through to form the sample beam LS. In the arrangement of this configuration, the reference beam LR is focused on the end facet 11 of the optical fiber 10 by the first GRIN lens 20, while the sample beam LS is focused on the sample 9 by the second GRIN lens 30 and is reflected by the sample 9, traveling through the second GRIN lens 30, the joint surface 50c and the first GRIN lens 20 sequentially, and then focused on the end facet 11 of the optical fiber 10. In addition, according to required energy of the sample beam LS, a reflectivity of the joint surface 50c may be designed to be larger than or equal to 4% and smaller than or equal to 50%, and for example, the joint surface 50c may be coated with a coating layer having a reflectivity larger than or equal to 4% and smaller than or equal to 50%. The present disclosure is not limited to the above described reflectivity of the joint surface 50c.

In order for the OCT common-path probe 1 to have a high light beam collection efficiency, material characteristics and lengths of the first GRIN lens 20, the second GRIN lens 30 and the rod lens 40 (if there is one) need to be calculated and designed, so that the maximum cross-section of the light beam LF in light traveling path is located at the joint surface 50, and focusing spots of the reference beam LR and the sample beam LS are located at the end facet 11 of the optical fiber 10.

The following paragraphs introduce multiple examples where refractive index gradient constants (self-focusing constant) of the first GRIN lens 20 and the second GRIN lens 30 of the OCT common-path probe 1 are the same, and ranges of optical parameters and condition settings of these examples.

Firstly, the ranges of optical parameters and condition settings include:
1. the refractive index gradient constant of the first GRIN lens 20 is identical to the refractive index gradient constant of the second GRIN lens 30, and the refractive index gradient constant thereof is larger than or equal to 1 and smaller than or equal to 3;
2. a ratio of the length of the first GRIN lens 20 to the length of the second GRIN lens 30 is larger than or equal to 1.374 and smaller than or equal to 7.7143;
3. a pitch of the first GRIN lens 20 and the second GRIN lens 30 is P, and the following condition is satisfied: 0.0363≤P≤0.3344, wherein a total length of the first GRIN lens 20 and the second GRIN lens 30 is Z, a refractive index gradient constant of the first GRIN lens 20 and the second GRIN lens 30 is √A, and 2πP=(√A)×Z;
4. the wavelength of the light beam LF is 1310 nm;
5. a working distance WD from the OCT common-path probe 1 to the sample 9 is larger than or equal to 1 mm and smaller than or equal to 3 mm, and a diameter of a spot of the sample beam LS focusing on the sample 9 is larger than or equal to 10 μm and smaller than or equal to 28.8 μm;
6. a diameter of a spot of the sample beam LS focusing on the end facet 11 of the optical fiber 10 is smaller than or equal to 9 μm;
7. a diameter of a spot of the reference beam LR focusing on the end facet 11 of the optical fiber 10 is smaller than or equal to 9 μm; and
8. a diameter of a core of the optical fiber 10 is 9 μm.

Please refer to Table 1 to Table 3 below, which show examples designed based on the above described ranges of optical parameters and condition settings, wherein the refractive index gradient constant of the first GRIN lens 20 and the second GRIN lens 30 is √A, the working distance from the OCT common-path probe 1 to the sample 9 is WD, the diameter of the spot of the sample beam LS focusing on the sample 9 is Spot, a length of the rod lens 40 is RL, the length of the first GRIN lens 20 is GL1, the length of the second GRIN lens 30 is GL2, and the pitch of the first GRIN lens 20 and the second GRIN lens 30 is P.

TABLE 1
Both Refractive Index Gradient Constant of First
Grin Lens And Refractive Index Gradient Constant
of Second Grin Lens Are Equal to 1

| | Exam- | Exam- | Exam- | Exam- | Exam- | Exam- |
|---|---|---|---|---|---|---|
| Parameter | ple 1 | ple 2 | ple 3 | ple 4 | ple 5 | ple 6 |
| √A | 1 | 1 | 1 | 1 | 1 | 1 |
| WD (mm) | 1 | 1 | 2 | 2 | 3 | 3 |
| Spot (μm) | 10 | 12.8 | 10.8 | 23.4 | 16.2 | 28.8 |
| RL (mm) | 0.71 | 0 | 1.67 | 0 | 1.68 | 0.5 |
| GL1 (mm) | 0.92 | 1.57 | 0.51 | 1.57 | 0.51 | 1.08 |
| GL2 (mm) | 0.55 | 0.53 | 0.3 | 0.3 | 0.2 | 0.2 |
| GL1/GL2 | 1.67273 | 2.9623 | 1.7 | 5.2333 | 2.55 | 5.4 |
| GL1 + GL2 | 1.47 | 2.1 | 0.81 | 1.87 | 0.71 | 1.28 |
| (mm) | | | | | | |
| P | 0.23408 | 0.3344 | 0.129 | 0.2978 | 0.1131 | 0.204 |

Note:
GL1 + GL2 in Table 1 represents the shortest workable total length of the first GRIN lens 20 and the second GRIN lens 30 in each example. However, the length of the first GRIN lens 20 and the length of the second GRIN lens 30 may be respectively increased by an integral multiple of half of the pitch (0.5 pitch) according to actual design requirements.

TABLE 2
Both Refractive Index Gradient Constant of First
Grin Lens And Refractive Index Gradient Constant
of Second Grin Lens Are Equal to 2

| | Exam- | Exam- | Exam- | Exam- | Exam- | Exam- |
|---|---|---|---|---|---|---|
| Parameter | ple 1 | ple 2 | ple 3 | ple 4 | ple 5 | ple 6 |
| √A | 2 | 2 | 2 | 2 | 2 | 2 |
| WD (mm) | 1 | 1 | 2 | 2 | 3 | 3 |
| Spot (μm) | 10 | 28.8 | 10 | 28.8 | 16.2 | 28.8 |
| RL (mm) | 0.93 | 0.11 | 1.95 | 0.25 | 1.91 | 0.93 |
| GL1 (mm) | 0.23 | 0.67 | 0.11 | 0.54 | 0.12 | 0.23 |
| GL2 (mm) | 0.15 | 0.11 | 0.08 | 0.07 | 0.05 | 0.05 |
| GL1/GL2 | 1.53333 | 6.0909 | 1.375 | 7.7143 | 2.4 | 4.6 |
| GL1 + GL2 | 0.38 | 0.78 | 0.19 | 0.61 | 0.17 | 0.28 |
| (mm) | | | | | | |
| P | 0.12102 | 0.2484 | 0.0605 | 0.1943 | 0.0541 | 0.0892 |

Note:
GL1 + GL2 in Table 2 represents the shortest workable total length of the first GRIN lens 20 and the second GRIN lens 30 in each example. However, the length of the first GRIN lens 20 and the length of the second GRIN lens 30 may be respectively increased by an integral multiple of half of the pitch (0.5 pitch) according to actual design requirements.

TABLE 3
Both Refractive Index Gradient Constant of First
Grin Lens And Refractive Index Gradient Constant
of Second Grin Lens Are Equal to 3

| | Exam- | Exam- | Exam- | Exam- | Exam- | Exam- |
|---|---|---|---|---|---|---|
| Parameter | ple 1 | ple 2 | ple 3 | ple 4 | ple 5 | ple 6 |
| √A | 3 | 3 | 3 | 3 | 3 | 3 |
| WD (mm) | 1 | 1 | 2 | 2 | 3 | 3 |
| Spot (μm) | 10 | 28.8 | 12.8 | 28.8 | 16 | 28.8 |
| RL (mm) | 0.97 | 0.17 | 1.54 | 0.62 | 1.92 | 0.99 |
| GL1 (mm) | 0.1 | 0.36 | 0.065 | 0.155 | 0.054 | 0.1 |
| GL2 (mm) | 0.07 | 0.07 | 0.04 | 0.04 | 0.02 | 0.02 |
| GL1/GL2 | 1.43 | 5.14 | 1.86 | 4.43 | 2.45 | 5 |
| GL1 + GL2 | 0.17 | 0.43 | 0.1 | 0.19 | 0.076 | 0.12 |
| (mm) | | | | | | |
| P | 0.081 | 0.205 | 0.047 | 0.091 | 0.0363 | 0.0573 |

Note:
GL1 + GL2 in Table 3 represents the shortest workable total length of the first GRIN lens 20 and the second GRIN lens 30 in each example. However, the length of the first GRIN lens 20 and the length of the second GRIN lens 30 may be respectively increased by an integral multiple of half of the pitch (0.5 pitch) according to actual design requirements.

The following paragraphs introduce multiple examples where refractive index gradient constants of the first GRIN lens 20 and the second GRIN lens 30 of the OCT common-path probe 1 are different from each other, and ranges of optical parameters and condition settings of these examples.

Firstly, the ranges of optical parameters and condition settings include:
1. the refractive index gradient constant of the first GRIN lens 20 and the refractive index gradient constant of the second GRIN lens 30 are different from each other and respectively larger than or equal to 1 and smaller than or equal to 3;
2. one of the first GRIN lens 20 and the second GRIN lens 30 having a smaller refractive index gradient constant has a longer length;
3. the wavelength of the light beam LF is 1310 nm;
4. a working distance WD from the OCT common-path probe 1 to the sample 9 is larger than or equal to 1 mm and smaller than or equal to 3 mm, and a diameter of a spot of the sample beam LS focusing on the sample 9 is larger than or equal to 10 μm and smaller than or equal to 28.8 μm;
5. a diameter of a spot of the sample beam LS focusing on the end facet 11 of the optical fiber 10 is smaller than or equal to 9 μm;
6. a diameter of a spot of the reference beam LR focusing on the end facet 11 of the optical fiber 10 is smaller than or equal to 9 μm; and
7. a diameter of a core of the optical fiber 10 is 9 μm.

Please refer to Table 4 below, which shows examples designed based on the above described ranges of optical parameters and condition settings, wherein the refractive index gradient constant of the first GRIN lens 20 is √A1, the refractive index gradient constant of the second GRIN lens 30 is √A2, the working distance from the OCT common-path probe 1 to the sample 9 is WD, the diameter of the spot of the sample beam LS focusing on the sample 9 is Spot, a length of the rod lens 40 is RL, the length of the first GRIN lens 20 is GL1, and the length of the second GRIN lens 30 is GL2.

TABLE 4
Refractive Index Gradient Constant of First Grin Lens 20 Is Different
From Refractive Index Gradient Constant of Second Grin Lens 30

| | Exam- | Exam- | Exam- | Exam- | Exam- | Exam- |
|---|---|---|---|---|---|---|
| Parameter | ple 1 | ple 2 | ple 3 | ple 4 | ple 5 | ple 6 |
| √A1 | 1 | 1 | 2 | 2 | 3 | 3 |
| √A2 | 2 | 3 | 1 | 3 | 1 | 2 |
| WD (mm) | 3 | 1 | 3 | 3 | 1 | 1 |
| Spot (μm) | 16.2 | 10 | 16.2 | 16.2 | 16.2 | 10 |
| RL (mm) | 1.76 | 0.41 | 1.94 | 1.91 | 0.7 | 0.99 |
| GL1 (mm) | 0.49 | 1.16 | 0.12 | 0.12 | 0.14 | 0.1 |
| GL2 (mm) | 0.051 | 0.068 | 0.2 | 0.023 | 0.55 | 0.15 |

Note:
in Table 4, when the refractive index gradient constant √A of one GRIN lens is smaller, the length GL of the GRIN lens is longer. That is, when √A1 is smaller than √A2, GL1 is larger than GL2, and vice versa.

In the above embodiments, the selection and designed length RL of the rod lens 40 are to adjust the focusing positions of the sample beam LS and the reference beam LR. Therefore, if the lengths of the first GRIN lens 20 and the second GRIN lens 30 are sufficient to focus both the sample beam LS and the reference beam LR on the end facet 11 of the optical fiber 10, the rod lens 40 may not be arranged in the OCT common-path probes 1.

In the OCT common-path probes 1 of each of the examples in Table 1 to Table 4 above, it is ensured that the diameters of the spots of the sample beam LS and the reference beam LR on the end facet 11 of the optical fiber 10 are smaller than or equal to the diameter (i.e., 9 μm in this embodiment) of the core of the optical fiber 10, such that the optical fiber coupling efficiency is 75% or above, which is at least 5 times more compared with prior art.

The above examples are merely exemplary, and the present disclosure is not limited to the ranges of optical parameters and condition settings in these examples. In other embodiments of the disclosure, ranges of optical parameters and condition settings may be different from those introduced above, as long as a first GRIN lens and a second GRIN lens are cemented to each other in series, a joint surface between the first GRIN lens and the second GRIN lens provides a reflection and transmission effect (e.g., light beam splitting function) so as to form a reference beam and a sample beam, and the reference beam and the sample beam are both focused on an end facet of an optical fiber after being reflected. By the design of optical parameters, the OCT common-path probe as disclosed by the disclosure have a high light beam collection efficiency.

According to the OCT common-path probe as described above, the first GRIN lens and the second GRIN lens are cemented to each other in series, and the joint surface provides a reflection and transmission effect (e.g., light beam splitting function), such that the reference beam is focused by the first GRIN lens, and the second GRIN lens is only for the sample beam to travel through. Therefore, the lengths of the first GRIN lens and the second GRIN lens can be adjusted in design in accordance with the reference beam and the sample beam, so that the reference beam and the sample beam can be both focused on the end facet of the optical fiber after being reflected, thereby ensuring the reference beam and sample beam collection efficiency. As a result, stable and consistent tomographic images of tissues can still be provided under handheld or dynamic operations, which is helpful for medical staff to determine the states of tissues or samples more precisely, and saving more time and human resources.

The embodiments were chosen and described in order to best explain the principles of the disclosure and its practical applications, to thereby enable others skilled in the art to best utilize the disclosure and various embodiments with various modifications as are suited to the particular use contemplated. It is intended that the scope of the disclosure be defined by the following claims and their equivalents.

What is claimed is:

1. An optical coherence tomography common-path probe for identifying a sample, and the optical coherence tomography common-path probe comprising:
an optical fiber having an end facet, and the optical fiber configured to output a light beam through the end facet;
a first GRIN lens located on one side of the end facet, and the first GRIN lens configured to collimate the light beam; and
a second GRIN lens cemented to the first GRIN lens, the first GRIN lens located between the end facet of the optical fiber and the second GRIN lens, and the second GRIN lens configured to focus a sample beam;
wherein a joint surface is located between the first GRIN lens and the second GRIN lens, after the light beam output from the optical fiber is collimated by the first GRIN lens, a part of the light beam is reflected by the joint surface to form a reference beam, and another part of the light beam passes through the joint surface to form the sample beam entering the second GRIN lens; wherein the reference beam is focused on the end facet of the optical fiber by the first GRIN lens;
wherein the sample beam is focused on the sample by the second GRIN lens and is reflected by the sample to travel through the second GRIN lens, the joint surface and the first GRIN lens sequentially, and thereby focused on the end facet of the optical fiber.

2. The optical coherence tomography common-path probe according to claim 1, wherein the joint surface has a partial reflection region and a light transmission region;
wherein the partial reflection region is configured for a part of the light beam reaching the partial reflection region to pass through the joint surface, and for another part of the light beam reaching the partial reflection region to be reflected;
wherein the light transmission region is configured for the light beam reaching the light transmission region to pass through.

3. The optical coherence tomography common-path probe according to claim 2, wherein an area of the partial reflection region is larger than or equal to 20% of a cross-sectional area of the light beam on the joint surface, the area of the partial reflection region is smaller than the cross-sectional area of the light beam on the joint surface, and a reflectivity of the partial reflection region is larger than or equal to 4% and smaller than 100%.

4. The optical coherence tomography common-path probe according to claim 2, wherein the partial reflection region of the joint surface faces toward the end facet of the optical fiber, and an optical axis of the first GRIN lens and the second GRIN lens is perpendicular to the partial reflection region.

5. The optical coherence tomography common-path probe according to claim 1, wherein the joint surface has a total reflection region and a light transmission region, and an area of the total reflection region is smaller than a cross-sectional area of the light beam on the joint surface;
wherein a part of the light beam from the end facet travels to the total reflection region of the joint surface, and is reflected by the total reflection region of the joint surface to form the reference beam;
wherein another part of the light beam from the end facet passes through the light transmission region of the joint surface and enters the second GRIN lens to form the sample beam.

6. The optical coherence tomography common-path probe according to claim 5, wherein an area of the total reflection region is larger than or equal to 20% of the cross-sectional area of the light beam on the joint surface.

7. The optical coherence tomography common-path probe according to claim 5, wherein the total reflection region of the joint surface faces toward the end facet of the optical fiber, and an optical axis of the first GRIN lens and the second GRIN lens is perpendicular to the total reflection region.

8. The optical coherence tomography common-path probe according to claim 1, wherein the joint surface is a partial reflection surface;
wherein a part of the light beam from the end facet travels to the joint surface, and is reflected by the partial reflection surface to form the reference beam;
wherein another part of the light beam from the end facet passes through the joint surface to form the sample beam.

9. The optical coherence tomography common-path probe according to claim 8, wherein the partial reflection surface faces toward the end facet of the optical fiber, and a reflectivity of the joint surface is larger than or equal to 4% and smaller than or equal to 50%.

10. The optical coherence tomography common-path probe according to claim 1, further comprising a rod lens, wherein the rod lens is located between the first GRIN lens and the end facet of the optical fiber and connected to the first GRIN lens and the end facet of the optical fiber.

11. The optical coherence tomography common-path probe according to claim 1, wherein a maximum cross-section of the light beam in a light traveling path is located at the joint surface.

12. The optical coherence tomography common-path probe according to claim 1, wherein when a refractive index gradient constant of the first GRIN lens is equal to a refractive index gradient constant of the second GRIN lens, a ratio of a length of the first GRIN lens to a length of the second GRIN lens is larger than or equal to 1.374 and smaller than or equal to 7.7143; when the refractive index gradient constant of the first GRIN lens is different from the refractive index gradient constant of the second GRIN lens, one of the first GRIN lens and the second GRIN lens having a smaller refractive index gradient constant has a longer length.

13. The optical coherence tomography common-path probe according to claim 1, wherein a pitch of the first GRIN lens and the second GRIN lens is P, and the following condition is satisfied:
0.0363≤P≤0.3344, wherein a total length of the first GRIN lens and the second GRIN lens is Z, a refractive index gradient constant of the first GRIN lens and the second GRIN lens is √A, and 2πP=(√A)×Z.

14. The optical coherence tomography common-path probe according to claim 13, wherein each of a length of the first GRIN lens and a length of the second GRIN lens is increased by an integral multiple of half of the pitch.

15. The optical coherence tomography common-path probe according to claim 1, wherein a working distance from the optical coherence tomography common-path probe to the sample is larger than or equal to 1 mm and smaller than or equal to 3 mm, and a diameter of a spot of the sample beam focusing on the sample is larger than or equal to 10 μm and smaller than or equal to 28.8 μm.

16. The optical coherence tomography common-path probe according to claim 1, wherein a diameter of a spot of the sample beam focusing on the end facet of the optical fiber is smaller than or equal to 9 μm, and a diameter of a spot of the reference beam focusing on the end facet of the optical fiber is smaller than or equal to 9 μm.
